(19) United States
(12) Patent Application Publication
(10) Pub. No.: US 2026/0034038 A1
(43) Pub. Date: Feb. 5, 2026

(54) FERMENTATION-PRODUCED PROPIONATE POWDER

(71) Applicant: PURAC Biochem B.V., Gorinchem (NL)

(72) Inventors: Michael BRECH, Gorinchem (NL); Nicolas Jean Yves GUILBOT, Gorinchem (NL); Mugesh KUMARARAJAN, Gorinchem (NL); Roel Johannes Adrianus van der VELDEN, Gorinchem (NL); Tithira Tirangika WIMALASENA, Gorinchem (NL)

(73) Assignee: PURAC Biochem B.V., Gorinchem (NL)

(21) Appl. No.: 19/356,566

(22) Filed: Oct. 13, 2025

Related U.S. Application Data
(63) Continuation of application No. PCT/EP2024/056585, filed on Mar. 12, 2024.

(30) Foreign Application Priority Data
Apr. 14, 2023 (EP) 23168034.9
Oct. 13, 2023 (EP) 23203618.6

(51) Int. Cl. A61K 8/36; A21D 2/14; A61K 8/362; C12P 7/52

(52) U.S. Cl. CPC A61K 8/36; A21D 2/14; A61K 8/362; C12P 7/52; A61K 2800/524; A61K 2800/85

(57) ABSTRACT

The present invention provides a fermentation-produced propionate powder comprising 25-60 wt. % propionic acid equivalent, 3-16 wt. % acetic acid equivalent, 2-12 wt % succinic acid equivalent, 0.1-0.8 mol Ca²⁺ per mol of propionate, 0-1.0 mol Na⁺ per mol of propionate, and 0-1.0 mol K⁺ per mol of propionate. Methods of preparing a food or cosmetic product containing the fermentation-produced propionate powder are also provided.

### CROSS-REFERENCE TO RELATED APPLICATIONS

The present application is a continuation of International Application No. PCT/EP2024/056585, filed Mar. 12, 2024, which claims priority to European Patent Application No. 23168034.9, filed Apr. 14, 2023, and European Patent Application No. 23203618.6, filed Oct. 13, 2023, all of which are hereby incorporated by reference herein in their entireties.

### TECHNICAL FIELD OF THE INVENTION

The present invention relates to a fermentation-produced propionate powder that comprises calcium propionate and at least one of sodium propionate and potassium propionate.

The invention also provides a process of producing such a propionate powder, said process comprising incubating a suitable substrate with propionic acid producing micro-organisms while adding an alkalising agent to maintain pH within the range of 5.0 to 7.5, said alkalising agent including an alkaline water-soluble calcium salt and an alkaline water-soluble alkali metal salt.

The invention further relates to an alternative process of producing the aforementioned propionate powder, said process comprising blending at least two different fermentation-produced propionate powders selected from:
fermentation-produced calcium propionate powder comprising 4-7 mol/kg of calcium propionate;
fermentation-produced sodium propionate powder comprising 4-6.5 mol/kg of sodium propionate;
fermentation-produced potassium propionate powder comprising 4-6 mol/kg of potassium propionate.

The invention also relates to a method of preserving a food product or cosmetic product, said method comprising incorporation of the propionate powder of the present invention.

### BACKGROUND OF THE INVENTION

Preservatives extend the shelf life of baked goods by inhibiting the growth of rope bacteria and moulds. Rope bacteria (Bacillus pumilus) can be carried through the baking process and cause an unpleasant texture and odour inside yeast-raised products like bread. Moulds can contaminate products after baking and then grow on the outside surface. Mould contamination is considered a serious problem amongst bakers, and conditions commonly found in baking present near-optimal conditions for mould growth.

Acidity decreases the chance of spoilage. Most preservatives have acids as an active ingredient. Preservatives can be classified according to the kind of acid they contain: mineral acid, organic acid, or fatty acid. Mineral acids (like sulfuric, hydrochloric, and phosphoric) are the strongest acids and so have the greatest effect on pH. But they work only by lowering pH and do not contain any other inhibitors. Organic acids (like lactic, benzoic, and tartaric) have less effect on pH than do mineral acids but are better preservatives because they have an additional inhibitory activity. Fatty acids (like acetic, sorbic, and propionic) are a specific kind of organic acid with an even stronger inhibitory activity.

Calcium propionate is used as a preservative in a wide variety of products, including: bread, other baked goods, processed meat, whey, and other dairy products. Propionates prevent microbes from producing the energy they need, like benzoates do. However, unlike benzoates, propionates do not require an acidic environment.

WO 2010/097362 describes a method for manufacturing a mixed solution comprising propionate and acetate, which comprises the steps of
providing a fermentation product comprising propionate, acetate, and carbonate-related compounds,
adding acid to the fermentation product to obtain an acidified fermentation product comprising propionate and acetate with a pH in the range of 2.5 to below 8.

WO 2016/146721 describes a process for manufacturing propionate products through fermentation, comprising the steps of:
fermenting a carbon source selected from sugars and lactate in a fermentation medium by means of a propionic acid producing micro-organism to provide a first fermentation broth comprising a propionate salt,
recovering propionic acid producing micro-organism from the first fermentation broth,
subjecting the first fermentation broth from which propionic acid producing micro-organism have been recovered to a water removal step to form a first propionate salt product,
fermenting a carbon source comprising glycerol with the propionic acid producing micro-organism recovered from the first fermentation broth in the presence of an inorganic alkaline salt to provide a second fermentation broth comprising a propionate salt,
subjecting the second fermentation broth to a purification step comprising at least one precipitation step, to form a second propionate salt product.

WO 2019/108064 describes an organic acid preservative system comprising an acetate component and a propionate component, wherein the acetate component and the propionate component make up at least 85 wt. % of the total amount of carboxylic acids and carboxylic acid salts in the preservative system and wherein the propionate component makes up 12 to 50 wt. % of said total amount of carboxylic acids and carboxylic acid salts.

### SUMMARY OF THE INVENTION

The inventors have discovered that the quality of fermentation-produced propionate powders can be improved significantly by ensuring that part of the propionate contained therein is present in the form of calcium propionate and that other part of the propionate is present in the form of sodium propionate and/or potassium propionate.

Accordingly, a first aspect of the invention concerns a fermentation-produced propionate powder comprising:
25-60 wt. % propionic acid equivalent;
3-16 wt. % acetic acid equivalent;
2-12 wt. % succinic acid equivalent;
0.1-0.8 mol Ca²⁺ per mol of propionate;
0-1.0 mol Na⁺ per mol of propionate;
0-1.0 mol K⁺ per mol of propionate;
wherein:

$$0. 4 \leq ( [ Na + ] + [ K + ] ) / [ propionate ] \leq 1.4 ;$$

$$1. 1 \leq ( 2 \cdot [ {Ca}^{2 +} ] + [ {Na}^{+} ] + [ {K}^{+} ] ) / [ propionate ] \leq 2.8 ;$$

[Ca²⁺] representing the concentration of Ca²⁺ in mmol/g of powder;
[Na⁺] representing the concentration of Na⁺ in mmol/g of powder;
[K⁺] representing the concentration of K⁺ in mmol/g of powder;
[propionate] representing the concentration of propionate in mmol/g of powder.

The fermentation-produced propionate powders of the present invention are superior to fermentation-produced propionate powders in which the bulk of the propionate consists of either calcium propionate, sodium propionate or potassium propionate, especially in terms of taste, smell and/or flow properties.

A second aspect of the invention relates to a process of preparing the propionate powder of the present invention, said process comprising:
providing an aqueous fermentation medium comprising 30-200 g/L glucose equivalent of hydrolysed glucan;
inoculating the fermentation medium with a propionic acid producing micro-organism;
incubating the inoculated fermentation medium for at least 20 hours to produce a fermentate while adding an alkalising agent to maintain pH within the range of 5.0 to 7.5, said alkalising agent including an alkaline water-soluble alkalimetal salt selected from sodium salt, potassium salt and combinations thereof; and an alkaline water-soluble calcium salt.

A third aspect of the invention relates to an alternative process of preparing the propionate powder of the present invention, said process comprising blending at least two different fermentation-produced propionate powders selected from:
fermentation-produced calcium propionate powder comprising 4-7 mol/kg of calcium propionate;
fermentation-produced sodium propionate powder comprising 4-6.5 mol/kg of sodium propionate;
fermentation-produced potassium propionate powder comprising 4-6 mol/kg of potassium propionate.

A fourth aspect of the invention relates to a method of preserving a food product or cosmetic product, said method comprising incorporation of the propionate powder of the present invention in a concentration of 0.1-5% by weight of the food product or cosmetic product.

A fifth aspect of the invention relates to a food product or a cosmetic product that is obtained by the aforementioned method.

### DETAILED DESCRIPTION OF THE INVENTION

The invention provides a fermentation-produced propionate powder comprising:
25-60 wt. % propionic acid equivalent;
3-16 wt. % acetic acid equivalent;
2-12 wt. % succinic acid equivalent;
0.1-0.8 mol Ca²⁺ per mol of propionate;
0-1.0 mol Na⁺ per mol of propionate;
0-1.0 mol K⁺ per mol of propionate;

wherein:

$$0.4 \leq ( [ Na + ] + [ K + ] ) / [ propionate ] \leq 1.4 ;$$

$$1. 1 \leq ( 2 \cdot [ {Ca}^{2 +} ] + [ {Na}^{+} ] + [ {K}^{+} ] ) / [ propionate ] \leq 2.8 ;$$

[Ca²⁺] representing the concentration of Ca²⁺ in mmol/g of powder;
[Na⁺] representing the concentration of Na⁺ in mmol/g of powder;
[K⁺] representing the concentration of K⁺ in mmol/g of powder;
[propionate] representing the concentration of propionate in mmol/g of powder.

The term “propionate” as used herein encompasses propionic acid as well as its salts.

Likewise, the terms “acetate” and “succinate” also encompass both the acid forms and salt forms.

The term “glucan” as used herein refers to a linear or branched polymer consisting of glucose monomeric units. Examples of glucans include amylose, amylopectin, cellulose and glycogen.

Concentrations of hydrolysed glucan expressed as “glucose equivalent” refer to the total amount of glucose present in hydrolysed glucan, including both free glucose and glucose that is contained in hydrolysis fragments that have a degree of polymerisation of two or more. The amount of glucose equivalent present in a hydrolysed glucan can be determined by completely hydrolysing the hydrolysed glucan and measuring the glucose content.

The propionate powder of the present invention preferably contains 30-48 wt. %, more preferably 32-46 wt. %, most preferably 33-44 wt. % of propionic acid equivalent.

The propionate powder preferably contains 4-14 wt. % acetic acid equivalent, more preferably 4.3-10 wt. % acetic acid equivalent and most preferably 4.5-9.0 wt. % acetic acid equivalent

The propionate powder preferably contains 2.5-10 wt. % succinic acid equivalent, more preferably 3.0-8.5 wt. % succinic acid equivalent and most preferably 3.5-7.5 wt. % succinic acid equivalent.

The propionate powder preferably comprises 0.15-0.50 mol Ca²⁺ per mol of propionate, more preferably 0.17-0.42 mol Ca²⁺ per mol of propionate and most preferably 0.18-0.32 mol Ca²⁺ per mol of propionate.

The propionate powder preferably comprises 0-0.7 mol Na⁺ per mol of propionate, more preferably 0.10-0.60 mol Nat per mol of propionate and most preferably 0.15-0.55 mol Na⁺ per mol of propionate.

The propionate powder preferably comprises 0.3-0.9 mol K⁺ per mol of propionate, more preferably 0.35-0.85 mol K⁺ per mol of propionate and most preferably 0.40-0.82 mol K⁺ per mol of propionate.

According to a particularly preferred embodiment, the propionate powder contains 0.18-0.32 mol Ca²⁺ per mol of propionate, preferably 0.40-0.82 mol K⁺ per mol of propionate and 0.15-0.55 mol Na⁺ per mol of propionate.

The propionate powder preferably contains sodium, potassium and propionate in such concentrations that the following requirement is met: 0.5≤([Na⁺]+[K⁺])/[propionate]≤1.3.

More preferably: 0.6≤([Na⁺]+[K⁺])/[propionate]≤1.2. Most preferably: 0.7≤([Na⁺]+[K⁺])/[propionate]≤1.1.

The propionate powder preferably contains calcium, sodium, potassium and propionate in such concentrations that the following requirement is met 1.2≤(2. [Ca²⁺]+[Na⁺]+[K⁺])/[propionate]≤2.2. More preferably, 1.25≤(2. [Ca²⁺]+[Na⁺]+[K⁺])/[propionate]≤2.00. Most preferably, 1.30≤(2. [Ca²⁺]+[Na⁺]+[K⁺])/[propionate]≤1.80.

The propionate powder of the present invention preferably has a moisture content of less than 12 wt. %, more preferably a moisture content of 1-10 wt. %.

Propionate is preferably contained in the propionate powder in a concentration of 26-55 wt. % propionic acid equivalent, more preferably in a concentration of 26-50 wt. % propionic acid equivalent and most preferably in a concentration of 27-38 wt. % propionic acid equivalent.

The fermentation-produced propionate powder typically contains a residual amount of hydrolysed glucan that was applied as substrate during fermentation. Hydrolysed glucan is preferably contained in the propionate powder in a concentration of 2-30 grams glucose equivalent per kg, most preferably 3-15 grams glucose equivalent per kg.

The hydrolysed glucan in the propionate powder is preferably selected from hydrolysed amylose, hydrolysed amylopectin, hydrolysed cellulose, hydrolysed glycogen and combinations thereof. Most preferably the hydrolysed glucan comprises hydrolysed amylose and/or hydrolysed amylopectin. Both amylose and amylopectin are starch components.

The hydrolysed glucan preferably comprises at least one or more saccharides selected from glucose, maltose, isomaltose, maltotriose, maltotetraose and combinations thereof. According to a particularly preferred embodiment, the propionate powder contains 0.5-10 g/kg of one or more saccharides selected from glucose, maltose, isomaltose, maltotriose, maltotetraose and combinations thereof, most preferably 0.8 to 5 g/kg of one or more saccharides selected from glucose, maltose, isomaltose, maltotriose, maltotetraose and combinations thereof.

According to a preferred embodiment, the propionate powder of the present invention comprises DNA from a propionic acid producing micro-organism, preferably from a bacterium belonging to the genera Propionibacterium and Acidipropionibacterium, more preferably from a bacterium selected from Acidipropionibacterium acidipropionici, Propionibacterium freudenreichii, Propionibacterium shermanii, Acidipropionibacterium thoenii, Acidipropionibacterium jensenii and combinations thereof. The presence of DNA from a propionic acid producing micro-organism can suitably be determined using a PCR test.

The propionate powder of the present invention advantageously has a near neutral pH. Accordingly, in a preferred embodiment, dispersion of 100 grams of the propionate powder in 1 litre of water of 20° C. yields an aqueous liquid having a pH in the range of 6 to 8.

In accordance with a preferred embodiment, at least 80 wt. % of the particles of the propionate powder have a diameter of not more than 500 μm. More preferably, the at least 80 wt. % of the particles have a diameter in the range of 40 to 400 μm. The particle size distribution of the dried fermentate may suitably be determined by means of laser diffraction.

Another aspect of the invention relates to process of preparing a propionate powder according to the present invention, said process comprising:
providing an aqueous fermentation medium comprising 30-200 g/L glucose equivalent of hydrolysed glucan;
inoculating the fermentation medium with a propionic acid producing micro-organism;
incubating the inoculated fermentation medium for at least 20 hours to produce a fermentate while adding an alkalising agent to maintain pH within the range of 5.0 to 7.5, said alkalising agent including an alkaline water-soluble alkalimetal salt selected from sodium salt, potassium salt and combinations thereof; and an alkaline water-soluble calcium salt.

The aqueous fermentation medium preferably comprises 40-180 g/L, most preferably 50-150 g/L of glucose equivalent of hydrolysed glucan.

According to a particularly preferred embodiment, the hydrolysed glucan in the aqueous fermentation medium is selected from hydrolysed amylose, hydrolysed amylopectin, hydrolysed cellulose, hydrolysed glycogen and combinations thereof. Most preferably the hydrolysed glucan comprises hydrolysed amylose and/or hydrolysed amylopectin. Both amylose and amylopectin are starch components.

According to a preferred embodiment, the hydrolysed glucan is obtained by hydrolysis of rice starch, wheat starch, tapioca starch and/or potato starch. More preferably, the hydrolysed glucan is obtained by hydrolysis of rice starch and/or wheat starch. Most preferably, the hydrolysed glucan is obtained by hydrolysis of rice starch. Brown rice syrup is an example of a suitable source of hydrolysed rice starch.

In a particularly preferred embodiment of the invention, the hydrolysed glucan employed in the present process is a partially hydrolysed starch, and during incubation the partially hydrolysed starch is further hydrolysed by added enzymes. Preferably, glucose constitutes not more than 70 wt. %, more preferably 10-60 wt. % of the partially hydrolysed starch.

Examples of enzymes that may be added to the fermentation medium to further hydrolyse the partially hydrolysed starch include glucoamylase, pullulanase, alpha-amylase, beta-amylase and combinations thereof. According to a particularly preferred embodiment, the added enzyme includes glucoamylase.

The propionic acid producing micro-organism that is employed in the present process preferably belongs to the genus Acidipropionibacterium. Examples of propionic acid producing micro-organism that may be employed in the present process include Acidipropionibacterium acidipropionici, Acidipropionibacterium freudenreichii, Acidipropionibacterium shermanii, Acidipropionibacterium thoenii, Acidipropionibacterium jensenii and combinations thereof. More preferably, the propionic acid micro-organism used is Acidipropionibacterium acidipropionici.

The incubation step is preferably carried out under anaerobic conditions.

The fermentation medium is preferably incubated at a temperature in the range of 20 to 45° C., more preferably of 25 to 42° C. and most preferably of 30 to 40° C.

The duration of the incubation step is preferably in the range of 22 to 120 hours, more preferably in the range of 24 to 72 hours.

In order to achieve a high yield of propionate, it is advantageous to continue the incubation step until virtually all hydrolysed glucan has been digested. Accordingly, in a preferred embodiment, incubation is continued until the fermentation medium contains no more than 10 g/L more preferably no more than 5 g/L, more preferably 0.1 to 3 g/L and most preferably 0.3 to 2 g/L of free glucose.

The incubation step is preferably continued until the propionate concentration in the fermentation medium has increased to at least 100 mmol/L, more preferably to at least 150 mmol/L and most preferably to 250-300 mmol/L.

The alkalising agent that is employed to control pH includes (i) an alkaline water-soluble alkalimetal salt and (ii) an alkaline water-soluble calcium salt. These two different alkalising agents may be added to the fermentation medium simultaneously or sequentially. Preferably the alkalimetal salt and the calcium salt are added simultaneously.

The alkaline water-soluble alkalimetal salt is preferably selected from sodium hydroxide, potassium hydroxide and combinations thereof.

The alkaline water-soluble calcium salt is preferably calcium hydroxide.

According to a particularly preferred embodiment of the present process, the alkalising agent is added to the fermentation medium to maintain pH within the range of 5.5 to 7.4 more preferably within the range of 5.8 to 7.2.

In the present process the propionate powder is preferably produced by drying the fermentate, more preferably by drying the fermentate by means of spray drying, drum drying, freeze drying or combination thereof. Most preferably the drying is carried out by means of spray drying.

In accordance with a particularly preferred embodiment of the invention, the present process contains the additional steps of:
removing undissolved solids from the fermentate by centrifugation or filtration to produce a low solids fermentate; and
drying the low solids fermentate.

This embodiment of the present process offers the advantage that it yields a propionate powder with a very high propionate content. Furthermore the propionate powders to obtained provide the additional advantage of having a low mean dynamic buffer capacity.

In the present process, undissolved solids may be removed by solid-liquid separation techniques known in the art, such as centrifugation, filtration, decanting and hydrocyclones. More preferably, the undissolved solids are removed by means of centrifugation or filtration. Most preferably, the undissolved solids are removed by means of disc stack centrifugation.

The inventors have observed that the undissolved solids that are removed from the fermentate include a significant amount of calcium salts. As a result, the molar ratio of calcium cation to propionate is substantially reduced by the removal of undissolved solids.

The drying of the low solids fermentate or the concentrated low solids fermentate is preferably carried out by means of spray drying, drum drying, freeze drying or combination thereof. Most preferably the drying is carried out by means of spray drying.

Another aspect of the invention relates to an alternative process of preparing a propionate powder according to the present invention, said process comprising blending at least two different fermentation-produced propionate powders selected from:
fermentation-produced calcium propionate powder comprising 4-7 mol/kg of calcium propionate;
fermentation-produced sodium propionate powder comprising 4-6.5 mol/kg of sodium propionate;
fermentation-produced potassium propionate powder comprising 4-6 mol/kg of potassium propionate.

Each of the fermentation-produced propionate powder that are employed in the blending process, i.e. the calcium propionate powder, the sodium propionate powder and the sodium propionate powder, preferably is a neutralised propionic acid fermentate containing 25-60 wt. % of propionic acid equivalent, 3-16 wt. % acetic acid equivalent and 2-12 wt. % succinic acid equivalent.

The calcium propionate powder that is employed in the blending process preferably contains 0.4-1.5 mol Ca²⁺ per mol of propionate, more preferably 0.6-1.3 mol Ca²⁺ per mol of propionate.

The sodium propionate powder that is employed in the blending process preferably contains 0.9-2.1 mol Na⁺ per mol of propionate, more preferably 1.2-2.0 mol Na⁺ per mol of propionate.

The potassium propionate powder that is employed in the blending process preferably contains 0.8-2.0 mol K⁺ per mol of propionate, more preferably 1.1-1.8 mol K⁺ per mol of propionate.

The fermentation-produced propionate powders that are employed in the blending method are preferably prepared by a process comprising:
providing an aqueous fermentation medium comprising 30-200 g/L glucose equivalent of hydrolysed glucan;
inoculating the fermentation medium with a propionic acid producing micro-organism;
incubating the inoculated fermentation medium for at least 20 hours to produce a fermentate while adding an alkalising agent to maintain pH within the range of 5.0 to 7.5.

The alkalising agent used in the preparation of the calcium propionate powder is an alkaline water-soluble calcium salt, more preferably calcium hydroxide.

The alkalising agent used in the preparation of the sodium propionate powder is an alkaline water-soluble sodium salt, more preferably sodium hydroxide.

The alkalising agent used in the preparation of the potassium propionate powder is an alkaline water-soluble potassium salt, more preferably potassium hydroxide.

including an alkaline water-soluble alkalimetal salt selected from sodium salt, potassium salt and combinations thereof; and an alkaline water-soluble calcium salt

The propionic acid producing micro-organism that is employed in the production of the propionate powders preferably belongs to the genus Acidipropionibacterium. Examples of propionic acid producing micro-organism that may be employed in the present process include Acidipropionibacterium acidipropionici, Acidipropionibacterium freudenreichii, Acidipropionibacterium shermanii, Acidipropionibacterium thoenii, Acidipropionibacterium jensenii and combinations thereof. More preferably, the propionic acid micro-organism used is Acidipropionibacterium acidipropionici.

Another aspect of the invention relates to a method of preserving a food product or cosmetic product with the dried fermentate, said method comprising incorporation of the propionate powder of the present invention in a concentration of 0.1-5% by weight of the food product or cosmetic product, more preferably in a concentration of 0.2-2% by weight of the food product or cosmetic product.

The present method is particularly suited for preserving food products, especially bakery products and process meat products. Most preferably, the present method comprises addition of the dried fermentate to a bakery product.

Yet another aspect of the invention relates to a food product or a cosmetic product that is obtained by the present preservation method.

In a preferred embodiment, the product obtained by the preservation method is a food product, more preferably a food product selected from a bakery product and a processed meat product, most preferably a bakery product.

The invention is further illustrated by the following non-limiting examples.

### EXAMPLES

### Example 1

A calcium propionate-containing fermentate was prepared starting from a fermentation medium that contained brown rice syrup as the only fermentable carbohydrate source. The composition of the fermentation medium is shown in Table 1.

TABLE 1

| Compounds | Concentration [g/kg] |
|---|---|
| Glucose equivalent from rice syrup | 130 |
| Yeast extract (50% d.m.) | 12.5 |
| Vitamins and minerals | 0.21 |
| NaOH solution 28% w/v | 6 |
| Inoculum ¹ | 75 |
| Enzyme mixture ² | 1 |
| Water | Remainder |

¹ Contains Acidipropionibacterium acidipropionici
² Comprises glucoamylase, pullulanase, alpha-amylase and lysophospholipase

Water, vitamins and minerals were added to the fermenters and the trace element solution so obtained was in situ sterilized at 121° C. for 20 minutes.

Next, the fermenters were cooled down to ˜ 40° C. and an UHT-sterilised aqueous dilution of brown rice syrup and yeast extract was added to the fermenters. The trace element solution was added aseptically to the fermenters and the bottles that contained the solution were subsequently rinsed with sterile demi water to ensure that all trace elements were transferred to the fermenter.

Prior to inoculation, the pH of the fermentation media was adjusted to pH 6.9 using ammonia solution.

An ammonia solution and Ca(OH)₂ were used to control pH. The ammonia solution was added during the first 12 hours of fermentation. Once the ammonia solution was depleted, a switch was made towards the Ca(OH)₂ solution.

The fermentation was started by addition of the inoculum. After inoculation, the enzyme mixture was added aseptically to the fermenter and the bottle was subsequently rinsed with sterile demi water to ensure that all enzyme had been added.

The fermentation was carried out under sterile condition at pH 7.0, 35° C. and under constant agitation.

When the free glucose concentrations had reached 8 g/L (total glucose around 13 g/L), addition of base was stopped and the fermentation continued until pH 5.8 was reached. This procedure minimized carbonate formation, foaming during downstream processing and reduced the amount of solids in the final product.

Next, biomass was removed from the fermentate by disc stack centrifugation, following which the clarified fermentate was concentrated, sterilised and spray dried. The powder so obtained had good flow properties.

The spray dried fermentate was analysed. The results of the analyses are summarised in Table 2.

TABLE 2

| | Wt. % |
|---|---|
| Acetate | 12.2 |
| Propionate | 49.3 |
| Succinate | 6.7 |
| Calcium | 18.5 |
| pH at 10%(w/v) dilution | 6.9 |

### Example 2

A sodium propionate-containing fermentate was prepared in the same way as the calcium propionate-containing fermentate of Example 1, except that no biomass was removed and that NaOH instead of Ca(OH)₂ was used to control pH during fermentation.

The spray dried sodium propionate powder obtained by spray drying had poor flow properties. The results of the analyses are summarised in Table 3.

TABLE 3

| | Wt. % |
|---|---|
| Acetate | 5.5 |
| Propionate | 35.3 |
| Succinate | 5.8 |
| Sodium | 20.4 |
| pH at 10%(w/v) dilution | 8.3 |

### Example 3

A potassium propionate-containing fermentate was prepared in the same way as the calcium propionate-containing fermentate of Example 1, except that no biomass was removed and that KOH instead of Ca(OH)₂ was used to control pH during fermentation.

The spray dried potassium propionate powder obtained by spray drying had very poor flow properties. The results of the analyses are summarised in Table 4.

TABLE 4

| | Wt. % |
|---|---|
| Acetate | 4.2 |
| Propionate | 33.1 |
| Succinate | 3.3 |
| Potassium | 26.9 |
| pH at 10%(w/v) dilution | 7.2 |

### Example 4

Powder blends were prepared from the propionate-containing fermentates of Examples 1-3 on the basis of the formulations shown in Table 5.

TABLE 5

| | Wt. % | | | |
|---|---|---|---|---|
| | Blend 1 | Blend 2 | Blend 3 | Blend 4 |
| Calcium propionate | 50 | 50 | 30 | 30 |
| Sodium propionate | 50 | | 30 | 10 |
| Potassium propionate | | 50 | 40 | 60 |

The compositions of the blends are summarised in Table 6.

| | Blend | Blend | Blend | Blend |
|---|---|---|---|---|
| | 1 | 2 | 3 | 4 |
| Calcium (mol/kg) | 2.3 | 2.3 | 1.4 | 1.4 |
| Sodium (mol/kg) | 4.5 | | 2.7 | 0.9 |
| Potassium (mol/kg) | | 3.5 | 2.8 | 4.1 |
| Propionate (mol/kg) | 5.7 | 5.6 | 5.2 | 5.2 |
| ([Na⁺] + [K⁺])/[propionate] | 0.8 | 0.6 | 1.0 | 1.0 |
| (2.[Ca²⁺] + [Na⁺] + [K⁺])/[propionate] | 1.6 | 1.4 | 1.4 | 1.4 |

All four powder blends had acceptable flow properties, the flow properties of blends 1 and 2 being better than those of blends 3 and 4.

The powder blends as well as the propionate powders used in these blends were dispersed in tap water (5 g/L). Next, the solutions were blindly evaluated by an expert panel.

The results of the sensory analyses are summarised in Table 6 (the higher the score, the higher the intensity of the taste attribute).

TABLE 6

| Powder | Bitter | Salty | Sweet | Astringent | Sour |
|---|---|---|---|---|---|
| Ca propionate | 43 | 9 | 24 | 25 | 12 |
| Na propionate | 37 | 11 | 28 | 19 | 10 |
| K propionate | 37 | 8 | 28 | 18 | 7 |
| Blend 1 | 39 | 9 | 30 | 22 | 10 |
| Blend 2 | 38 | 8 | 26 | 21 | 11 |
| Blend 3 | 37 | 7 | 32 | 19 | 6 |
| Blend 4 | 38 | 7 | 29 | 20 | 7 |

The panellists commented that the calcium propionate powder was very bitter, astringent and sour. They further commented that the sodium propionate powder had a burned, potato-like flavour.

### Example 5

A spray dried propionate-containing fermentate (Ca/Na propionate powder) was prepared in the same way as the calcium propionate-containing fermentate of Example 1, except that no biomass was removed and that instead of Ca(OH)₂ a mixture of Ca(OH) 2/NaOH (60:40 w/w) was used to control pH during fermentation.

Another spray dried propionate-containing fermentate (Ca propionate powder) was prepared in the same way as the calcium propionate-containing fermentate of Example 1, except that no biomass was removed.

Both spray dried propionate powders had good flow properties. The results of the analyses of the two powders are summarised in Table 7.

TABLE 7

Wt. %

| | Ca/Na | Ca |
|---|---|---|
| | propionate | propionate |
| Acetate | 7.2 | 7.4 |
| Propionate | 31.5 | 30.0 |
| Succinate | 5.7 | 4.3 |
| Calcium | 11 | 18 |
| Sodium | 9.4 | 1 |
| pH at 10%(w/v) dilution | 6.3 | 6.6 |

Both the Ca/Na propionate powder and the Ca propionate powder were dissolved in water at a concentration of 5% w/v. Ten untrained panellists evaluated each sample. The results of the evaluation are shown in Table 8. This table shows the mean scores for attribute Ranking Test using an anchored line scale labelled with “low” on the one end and “high” on the opposite end.

TABLE 8

| | Bitter | Sweet | Astringent | Powdery |
|---|---|---|---|---|
| Solution of | Intensity | Intensity | Intensity | Intensity |
| Ca/Na propionate powder | 32.9 | 32.25 | 29.3 | 30.2 |
| Ca propionate powder | 45.6 | 26.8 | 28.4 | 30.5 |

### Example 6

The propionate powders of Example 5 were applied in a typical baked good, bread. The bread recipes are shown in Table 9 (ferments were added in amount providing 3.9 grams of propionic acid).

TABLE 9

Grams

| | Bread 1 | Bread A |
|---|---|---|
| Flour | 1000.00 | 1000.00 |
| Salt | 20.00 | 20.00 |
| Sugar | 80.00 | 80.00 |
| Anti-staling enzymes¹ | 5.00 | 5.00 |
| Dough conditioner² | 3.25 | 3.25 |
| Yeast - compressed | 120.00 | 120.00 |
| Soy oil | 20.00 | 20.00 |
| 60% lactic acid solution | 8.90 | 10.30 |
| Water | 566.00 | 566.00 |
| Ca/Na propionate powder | 12.38 | |
| Ca propionate powder | | 13.00 |

¹Anti-staling enzymes can include ULTRAFRESH Premium 250, available from Corbion
²Dough conditioner can include PRISTINE Concentrate and PRISTINE Relaxer available from Corbion

The breads were prepared by the following process:
1. Mix dry ingredients
2. Add soy oil and lactic acid solution to dry ingredient mixture
3. Chill the water and add to mixing bowl
4. Add dry ingredients with soy oil to chilled water
5. Add yeast
6. Mix for 2 minutes on low speed
7. Mix 11-13 minutes on medium speed
8. Cover doughs with a towel and rest for 5 minutes
9. Cut into 6 portions, round, and allow to rest for another 5 minutes
10. Sheet, roll, and place into greased baking pan
11. Place in proofing box until dough height reaches proofing template
12. Bake at 420° F. (216° C.) for 23 minutes
13. Cool for 1 hour
14. Bag and seal

28 untrained panellists evaluated each bread sample for various aspects of liking. Results are shown in Table 10.

TABLE 10

| | Overall | Aroma | Flavour | After-taste |
|---|---|---|---|---|
| Bread 1 | 5.8 | 6.2 | 5.9 | 5.1 |
| Bread A | 4.9 | 5.7 | 5.0 | 4.9 |

Notes regarding the testing:
1. The testing used a questionnaire with a 9-point scale where 9=Like extremely and 1=Dislike extremely and 5=Neither like nor dislike
2. Samples were tasted in pairs with Bread Samples 1 & A compared to each other and Bread Samples 2 & B compared to each other

1. A fermentation-produced propionate powder comprising:
25-60 wt. % propionic acid equivalent;
3-16 wt. % acetic acid equivalent;
2-12 wt. % succinic acid equivalent;
0.1-0.8 mol Ca²⁺ per mol of propionate;
0-1.0 mol Na⁺ per mol of propionate; and
0-1.0 mol K⁺ per mol of propionate;
wherein:
$$0.4 \leq ( [ Na + ] + [ K + ] ) / [ propionate ] \leq 1.4 ; and$$
$$1. 1 \leq ( 2 [ {Ca}^{2 +} ] + [ {Na}^{+} ] + [ {K}^{+} ] ) / [ propionate ] \leq 2.8 ,$$
wherein [Ca²⁺] represents the concentration of Ca²⁺ in mmol/g of powder;
[Na⁺] represents the concentration of Na⁺ in mmol/g of powder;
[K⁺] represents the concentration of K⁺ in mmol/g of powder; and
[propionate] represents the concentration of propionate in mmol/g of powder.

2. The fermentation-produced propionate powder according to claim 1, wherein the powder comprises 30-48 wt. % propionic acid equivalent.

3. The fermentation-produced propionate powder according to claim 1, wherein the powder comprises 0.15-0.50 mol Ca²⁺ per mol of propionate.

4. The fermentation-produced propionate powder according to claim 1, wherein the powder comprises 0.3-0.9 mol K⁺ per mol of propionate.

5. The fermentation-produced propionate powder according to claim 1, wherein the powder comprises 0-0.7 mol Na⁺ per mol of propionate.

6. The fermentation-produced propionate powder according to claim 1, wherein the powder comprises at least 0.1 mol Na⁺ per mol of propionate.

7. The fermentation-produced propionate powder according to claim 1, wherein:
$$0.5 \leq ( [ Na + ] + [ K + ] ) / [ propionate ] \leq 1.1 .$$

8. The fermentation-produced propionate powder according claim 1, wherein:
$$1.2 \leq ( 2 [ {Ca}^{2 +} ] + [ {Na}^{+} ] + [ {K}^{+} ] ) / [ propionate ] \leq 2.2 .$$

9. A process of preparing a fermentation-produced propionate powder according to claim 1, the process comprising:
providing an aqueous fermentation medium comprising 30-200 g/L glucose equivalent of hydrolysed glucan;
inoculating the fermentation medium with a propionic acid producing micro-organism; and
incubating the inoculated fermentation medium for at least 20 hours to produce a fermentate while adding an alkalising agent to maintain pH within the range of 5.0 to 7.5, said alkalising agent including an alkaline water-soluble alkalimetal salt selected from sodium salt, potassium salt and combinations thereof; and an alkaline water-soluble calcium salt.

10. The process according to claim 9, wherein the propionic acid producing micro-organism belongs to the genus Acidipropionibacterium.

11. The process according to claim 9, wherein the alkaline water-soluble alkalimetal salt is selected from sodium hydroxide, potassium hydroxide and combinations thereof and wherein the alkaline water-soluble calcium salt is calcium hydroxide.

12. A process of preparing a propionate powder according to claim 1, the process comprising blending at least two different fermentation-produced propionate powders selected from:
fermentation-produced calcium propionate powder comprising 4-7 mol/kg of calcium propionate;
fermentation-produced sodium propionate powder comprising 4-6.5 mol/kg of sodium propionate; and
fermentation-produced potassium propionate powder comprising 4-6 mol/kg of potassium propionate.

13. The process according to claim 12, wherein each of the calcium propionate powder, the sodium propionate powder, and the sodium propionate powder is a neutralised propionic acid fermentate containing 25-60 wt. % of propionic acid equivalent, 3-16 wt. % acetic acid equivalent, and 2-12 wt. % succinic acid equivalent.

14. A method of preserving a food product or cosmetic product, the method comprising incorporation of a propionate powder according to claim 1 in a concentration of 0.1-5% by weight of the food product or cosmetic product.

15. A food product or a cosmetic product obtained by the method according to claim 14.
